(19) United States
(12) Patent Application Publication
(10) Pub. No.: US 2024/0335622 A1
(43) Pub. Date: Oct. 10, 2024

(54) SUPPORT DEVICE FOR MEDICATION DELIVERY DEVICE

(71) Applicant: Becton, Dickinson and Company, Franklin Lakes, NJ (US)

(72) Inventors: Rajasekara PRABHU, Thiruchuli (IN); Xavier Poncruz XAVIER PONCRUZ PAUL, Theni (IN)

(73) Assignee: Becton, Dickinson and Company, Franklin Lakes, NJ (US)

(21) Appl. No.: 18/293,191

(22) PCT Filed: Aug. 2, 2021
(86) PCT No.: PCT/US2021/044129

§ 371 Date: Jan. 29, 2024

(51) Int. Cl. A61M 5/42; A61M 5/46

(52) U.S. Cl. CPC A61M 5/425; A61M 5/46

(57) ABSTRACT

A support (10) for a delivery device, such as a syringe (18) or pen needle delivery device, includes an adapter (12), a guide (16) for coupling to the adapter, and holder (14) for the delivery device that is able to slide relative to the adapter. The adapter includes movable arms (30) for gripping or pinching the tissue of the patient into raised, bulged area. The guide (16) is coupled to the adapter between the arms of the adapter to guide the delivery device into a position where the needle can access the tissue between the arms. The holder for the delivery device slides in the guide to guide the needle of the delivery device into the tissue between the arms to a controlled depth for introducing the medication to the patient.

### FIELD OF THE INVENTION

The present invention relates to a medical device, and more particularly, to a support for a delivery device for delivering a medication into a patient. The support is suitable for a needle injection delivery device where the support is configured for contacting the surface of the skin of a patient for the subcutaneous delivery of a medication, such as insulin.

### BACKGROUND OF THE INVENTION

Diabetes is a group of diseases characterized by high levels of blood glucose resulting from the inability of diabetic patients to maintain proper levels of insulin production when required. Diabetes can be dangerous to the affected patient if it is not treated, and it can lead to serious health complications and premature death. However, such complications can be minimized by utilizing one or more treatment options to help control the diabetes and reduce the risk of complications.

The treatment options for diabetic patients include specialized diets, oral medications and/or insulin therapy. The main goal of diabetes treatment is to control the diabetic patient's blood glucose or sugar level. However, maintaining proper diabetes management may be complicated because it has to be balanced with the activities of the diabetic patient. Type 1 diabetes (T1D) patients are required to take insulin (e.g., via injections or infusion) to move glucose from the bloodstream because their bodies generally cannot produce insulin. Type 2 diabetes (T2D) patients generally can produce insulin but their bodies cannot use the insulin properly to maintain blood glucose levels within medically acceptable ranges. In contrast to people with T1D, the majority of those with T2D usually do not require daily doses of insulin to survive. Many people are able to manage their condition through a healthy diet and increased physical activity or oral medication. However, if they are unable to regulate their blood glucose levels, they will be prescribed insulin. For example, there are an estimated 6.2 million Type 2 diabetes patients (e.g., in the United States, Western Europe and Canada) taking multiple-daily-injections (MDI) which consist of a 24-hour basal insulin and a short acting rapid insulin that is taken at mealtimes for glycemic management control.

For the treatment of Type 1 diabetes (TID) and sometimes Type 2 diabetes (T2D), there are two principal methods of daily insulin therapy. In the first method, diabetic patients use syringes or insulin pens to self-inject insulin when needed. This method requires a needle stick for each injection, and the diabetic patient may require three to four injections daily. The syringes and insulin pens that are used to inject insulin are relatively simple to use and cost effective.

An effective method for insulin therapy and managing diabetes is infusion therapy or infusion pump therapy in which an insulin pump is used. The insulin pump is able to provide continuous infusion of insulin to a diabetic patient at varying rates to more closely match the functions and behavior of a properly operating pancreas of a non-diabetic person that produces the required insulin, and the insulin pump can help the diabetic patient maintain his/her blood glucose level within target ranges based on the diabetic patient's individual needs. Infusion pump therapy requires an infusion cannula, typically in the form of an infusion needle or a flexible catheter, that pierces the diabetic patient's skin and through which infusion of insulin takes place. Infusion pump therapy offers the advantages of continuous infusion of insulin, precision dosing, and programmable delivery schedules.

Other common methods of delivery of insulin include a syringe or injection pen to provide a prescribed dosage to the patient. Syringe injection or injection pen delivery can be required several times a day to adequately maintain proper blood sugar levels. One procedure for the self-administration of insulin by a syringe or pen needle requires the patient to manually pinch a surface area of the skin to form a raised pinched area where the injection is intended to take place. The injection procedure requires the patient to pinch the skin with one hand while handling the syringe or pen needle with the other hand during the insertion of the needle and delivery of the insulin. This can result in the needle not injecting the insulin to a desired depth or misalignment of the needle relative to the surface of the skin during the injection.

Prior devices are known for pinching or gathering the surface of the skin of a patient for inserting a needle into the patient. However, these prior devices do not provide the controlled pinching of the skin or controlled alignment of the delivery device relative to the surface of the skin to provide consistent delivery of a medication, such as insulin.

Accordingly, a need exists for an improved device to assist the patient in positioning the delivery device and needle at a desired depth and for delivering the intended dosage at the intended location in the patient.

### SUMMARY

The present device is for use in assisting a patient for the delivery of a medication, such as insulin. The device is particularly suitable for the self-injection of insulin or other medication by a patient to provide a consistent depth of injection.

The device as disclosed herein is a support assembly for use in conjunction with a delivery device. The delivery device in the embodiments described include a syringe having a needle or cannula for injecting the medication. In one embodiments, the delivery device can be a syringe or a pen needle delivery device having a needle or cannula for injecting the medication. The device is configured to support the delivery device and position or orient the delivery device relative to the surface of the tissue or skin of the patient to assist in the consistent delivery and depth of penetration of the needle or cannula during an injection.

The support in one embodiment is able to grip and/or pinch the surface of the skin or tissue of the patient prior to and during an injection. The support includes at least two arms that can move toward each other toward a center axis of the support for gathering or pinching the tissue to form a bulge or raised surface of the tissue that is accessible for injecting the medication. The arms of the support are biased inwardly toward each other and toward the center axis of the support. The arms can include a finger tab or grip that can be pressed or pulled together to open the arms outwardly for engaging the tissue. The arms are biased to close the gap between the arms to pinch the tissue between the arms.

In one embodiment, the support includes an adapter, a guide, and a holder for the delivery device. The adapter can be a one-piece unitary member that can be made of a plastic material or spring steel. The adapter can have a body portion with distally extending arms that are biased toward each other. The arms can biased by the spring steel of the adapter. The body can include gripping tabs extending in a proximal direction opposite the arms for opening or spreading the arms during use.

The adapter in one embodiment has a central opening extending axially between the arms for receiving the needle or cannula of the delivery device so that the needle or cannula can pierce the tissue in an orientation substantially perpendicular to the surface of the tissue captured between the arms. A holder for the delivery device is coupled to the adapter and is configured to slide in an axial direction relative to the adapter for moving the delivery device toward the tissue of the patient for insertion of the needle or cannula into the tissue of the patient at a controlled depth.

In one embodiment, the support includes a holder that is able to receive a delivery device, such as a syringe barrel or pen needle delivery device, and slide the syringe barrel or pen needle delivery device in a linear direction relative to the support to insert the needle into the patient. The holder can slide outwardly from the support to withdrawn the needle or cannula from the patient. The adapter can then be released from the tissue of the patient.

The features of the device are basically attained by providing a support for an injection delivery device including an adapter having at least two movable arms, where each arm has a proximal end pivotally movable relative to each other, The arms have a distal end configured for engaging and pinching tissue of a patient to form a raised area of the tissue. A guide is coupled to the adapter and a holder is coupled to said guide. The holder is configured for supporting the injection delivery device and for sliding the injection delivery device in a distal direction relative to the adapter to deliver a medication to the tissue pinched between the arms. The delivery device typically has a needle extending in a distal direction for piercing the tissue that is pinched between the arms where the medication is delivered to the tissue between the arms. In one embodiment, the adapter is a one-piece unitary structure made of a spring material to spring the arms to a position where the tissue is pinched between the arms.

The features of the device include a support for an injection delivery device for delivering a medication to a patient, where the support includes an adapter having a body with an axial passage and two arms extending in a distal direction from the body. The adapter in one embodiment is a one-piece, unitary member where the arms are biased inwardly toward each other and are configured for pinching and gathering tissue of a patient between the arms to form a raised surface area on the tissue. A guide is removably coupled to said adapter. The guide has an axial passage aligned with the axial passage of the adapter. A holder is coupled to the guide, where the holder has a coupling member configured for coupling to the injection delivery device. The holder is configured for sliding the injection delivery device in an axial direction relative to the guide and adapter for introducing a needle of the injection delivery device into the tissue between the arms.

The features of the support are also provided by a method of injecting a medication into subcutaneous tissue of a patient. The method positions a support against a patient, where the support includes an adapter having a body with an axial passage and two arms extending in a distal direction from the body. The adapter is a one-piece, unitary member. The arms are biased inwardly toward each other and are configured for pinching tissue of a patient between the arms. A guide is removably coupled to the adapter. The guide has an axial passage aligned with the axial passage of the adapter. A holder is coupled to the guide, where the holder has a coupling member configured for coupling to the injection delivery device. The holder is configured for sliding the injection delivery device in an axial direction relative to the guide and the adapter for introducing a needle of the injection delivery device into the tissue between the arms. The method includes the steps of expanding the arms of the adapter outwardly and releasing the arms to pinch the subcutaneous tissue of the patient to form a pinched surface, coupling the injection delivery device into the holder and guide, coupling the holder and guide to the adapter, sliding the holder and the injection device relative to the guide and adapter to insert the needle into the subcutaneous tissue, and delivering the medication to the patient.

Additional and/or other aspects and advantages of the present delivery device is set forth in the description that follows or will be apparent from the description, or may be apparent by practice of the invention. The present invention may comprise delivery devices and methods for forming and operating same having one or more of the above aspects, and/or one or more of the features and combinations thereof. The present invention may comprise one or more of the features and/or combinations of the above aspects as recited, for example, in the attached claims.

### BRIEF DESCRIPTION OF THE DRAWINGS

The various aspects and advantages of embodiments of the delivery device will be more readily appreciated from the following detailed description, taken in conjunction with the accompanying drawings, of which

FIG. 1 is a perspective view of the support assembly and a delivery device in accordance with an illustrative embodiment;

FIG. 2 is front perspective view of the support assembly device of FIG. 1;

FIG. 3 is an exploded front view of the support assembly of FIG. 2;

FIG. 4 is an exploded rear view of the support assembly;

FIG. 5 is a right side view of the support assembly;

FIG. 6 is a left side view of the support assembly;

FIG. 7 is a rear view of the support assembly;

FIG. 8 is a front view of the support assembly;

FIG. 9 is a top view of the support assembly;

FIG. 10 is bottom view of the support assembly;

FIG. 11 is a cross-sectional side view of the support assembly;

FIG. 12 is a front view of the support assembly showing the delivery device in the ready position;

FIG. 13 is a front view of the support assembly showing the delivery device in the injection position; and

FIG. 14 is a front view of the support assembly in a further embodiment.

### DETAILED DESCRIPTION OF EMBODIMENTS

Reference will now be made in detail to embodiments of the present invention, which are illustrated in the accompanying drawings, wherein like reference numerals refer to like elements throughout. The embodiments described herein exemplify, but do not limit, the present invention by referring to the drawings.

It will be understood by one skilled in the art that this disclosure is not limited in its application to the details of construction and the arrangement of components set forth in the following description or illustrated in the drawings. The embodiments herein are capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting. Any of the embodiments and/or elements and features disclosed herein may be combined with one another to form various additional embodiments not specifically disclosed, as long as they do not contradict or are not inconsistent with each other. Terms of degree, such as “substantially”, “about” and “approximately” are understood by those skilled in the art to refer to reasonable ranges around and including the given value and ranges outside the given value, for example, general tolerances associated with manufacturing, assembly, and use of the embodiments. The term “substantially” when referring to a structure or characteristic includes the characteristic that is mostly or entirely.

The illustrative embodiments are described with reference to injecting a medication into a patient at a selected depth. It is to be understood that these illustrative embodiments can be used with different drug therapies and regimens to treat physiological conditions other than diabetes using different medicaments. In the embodiment described, the device is configured for insulin delivery using a syringe and needle although it is understood that the disclosure is not limited to a syringe as the delivery device.

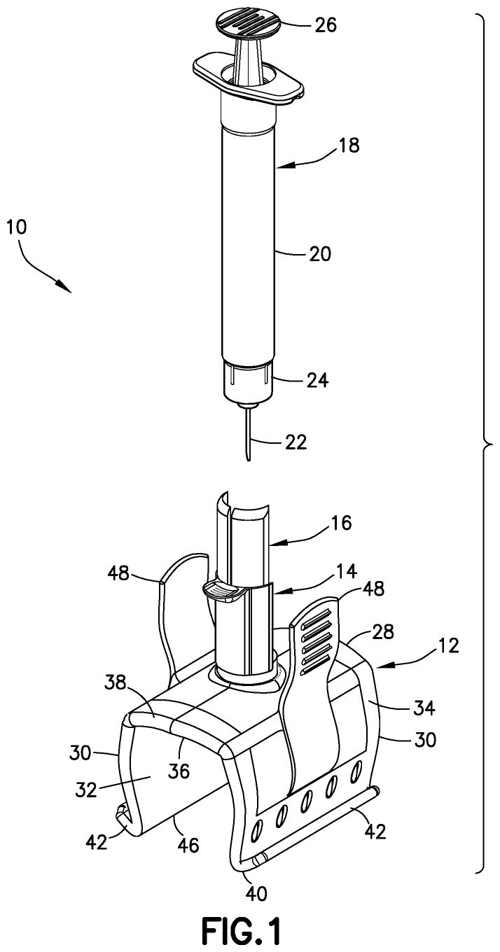

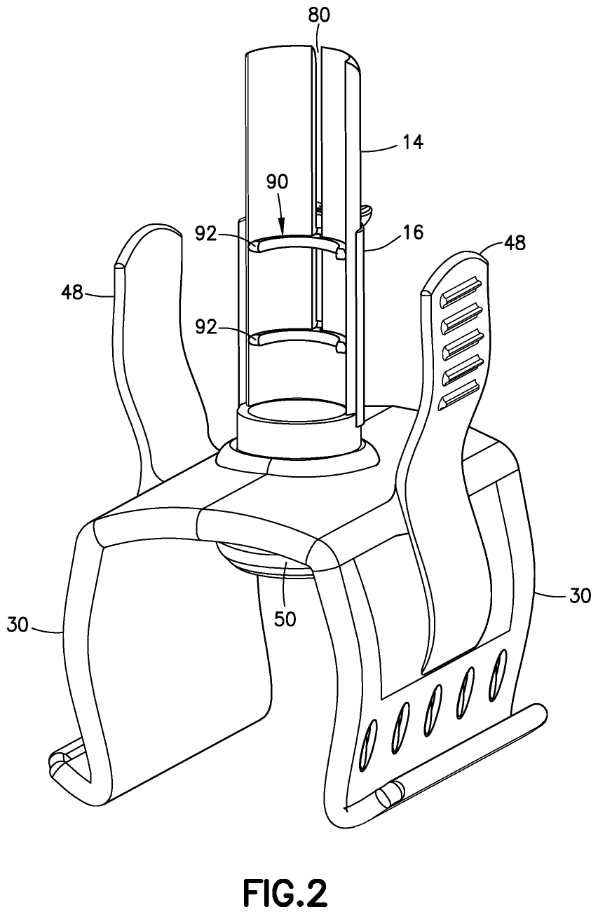

The device as described herein include a support assembly 10 for assisting in injecting a medication into a patient in a controlled manner. The support assembly 10 includes an adapter 12, a guide 14 and a holder 16. The assembly 10 as shown in FIG. 1 and FIG. 2 illustrate the adapter 12, the guide 14 and the holder 16 in an assembled condition ready for use for injecting a medication to the patient. The support assembly is particularly suitable for injecting a prescribed dosage of insulin to a patient although the device can be used for other medications. The following description refers to the delivery or injection of insulin, although it is understood that the assembly can be used for other medications.

The support assembly 10 is constructed for receiving and supporting an injection delivery device for injecting the medication into the patient. The injection delivery device as shown in the drawings as a syringe 18 having a syringe barrel 20, a needle 22 extending from a distal end 24 of the barrel 20, and a plunger 26. In the embodiment shown, the distal end of the syringe barrel 20 includes a hub 28 for supporting the needle 22. The hub 28 in the embodiment shown has an outer diameter less than an outer diameter of the syringe barrel 20. The syringe barrel 20 has a cylindrical shaped sidewall with an open proximal end as known in the art. In other embodiments, the injection delivery device can be an injection pen such as a pen needle injection device or injection pen as known in the art. The following disclosure refers generally to a syringe for injecting the medication, such as insulin, into the patient although the support assembly can be used interchangeably with an injection pen.

Referring to the drawings, the adapter 12 is configured for supporting the injection device such as a syringe 18 during use and delivery and injection of the insulin or other medication. In the embodiment shown, the adapter 12 includes a body 28 with a distal side and a proximal side. Arms 30 extend distally from the body 28 and have a shape and dimension for gripping and/or pinching the tissue of a patient and deforming the tissue to assist in injecting the medication to a desired depth. The body 28 has a width to accommodate and support the syringe 18. The arms have a width to grip a surface area of the tissue of the patient for the injection. In the embodiment shown, the body has a curved or arcuate shape although other suitable shapes can be provided.

The arms 30 extend from the outer side edges of the body 28 so that the arms are spaced apart a distance suitable for gripping sufficient surface area of the skin and/or tissue during the injection. The arms 30 are biased inwardly toward a center axis of the adapter 12. In the embodiment shown, the adapter 12 is formed as a one-piece unitary and integrally formed structure with the arms 30 formed with the body 28 as a single piece. The adapter 12 in the embodiment shown is made from a spring material so that the arms 30 are biased inwardly toward each other and can be moved outwardly for positioning on the surface of the skin or tissue of the patient and released to spring back to the original position. The adapter 12 can be made of suitable spring steel or a plastic material having sufficient flexibility where the arms 30 can hinge or bend outward and spring back to the original position and shape. In the embodiments shown, the adapter 12 includes two arms 30 spaced apart from each other and biased inwardly toward each other. In other embodiments, more than two arms can be used to provide the desired gripping force.

The arms 30 have a shape and configuration for gripping the tissue and springing back to the original shape to gather the tissue and from a bulged area on the surface of the tissue. As shown in the drawings, the arms 30 have a generally curved shape with a concave inner surface 32 and a convex outer surface 34. In the embodiment shown, the body 28 also has a generally curved shape with a concave bottom inner surface 36 and a convex top outer surface 38. The distal end 40 of the arms 30 are curved inwardly relative to a midpoint of the arms 30. The distal ends 40 of the arms 30 include an outwardly extending flange 42 forming a curved gripping surface 44 at the distal end 40 of the arms 30. In the embodiment shown, the flanges 42 are directed at an inclined angle outwardly from the arms 30 and in a generally proximal direction with respect to the body 28. A non-slip or friction surface 46 or member can be provided on the curved surface 44 to increase the gripping of the tissue during use. In the embodiment shown, the body 28 has a width greater than the width of the distal end of the arms and a curved gripping surface 44.

The arms 30 are positioned to define a gap between the distal ends 40 of the arms 30 which can be opened to enlarge the gap for pinching a surface area of the tissue of the patient. The arms 30 spring back and close to the original position shown in FIGS. 1 and 2 to define a gap having a width suitable to pinch an amount of the tissue and form a raised portion on the surface of the tissue for introducing the needle and injecting the medication from the delivery device.

The adapter 12 is provided with actuating members shown as tabs 48 extending proximally from the body 28 for opening the gap between the arms 30 and manipulating the adapter. The tabs 48 are formed at the outer edges of the body as shown and spaced apart a distance to allow the user to depress the tabs 48 inwardly toward each other to spread the distal end of the arms outwardly. In one embodiment, the tabs 48 are integrally formed with the arms 30 and the body 28 as a one-piece unitary unit. The tabs 48 have a generally curved shape and gripping member 50 to assist in the manipulation of the arms and the support assembly. The tabs 48 have a length sufficient to allow the user to easily grip and squeeze the tabs toward each other to open the gap between the arms and release the arms for pinching the tissue between the distal end of the arms. The tabs 48 are oriented and spaced from the guide 14 and holder 16 in the relaxed position a distance so that the tabs can move inwardly a distance to open the arms a distance to be able grip an amount of the tissue to form the intended raised area or bulge for the injection.

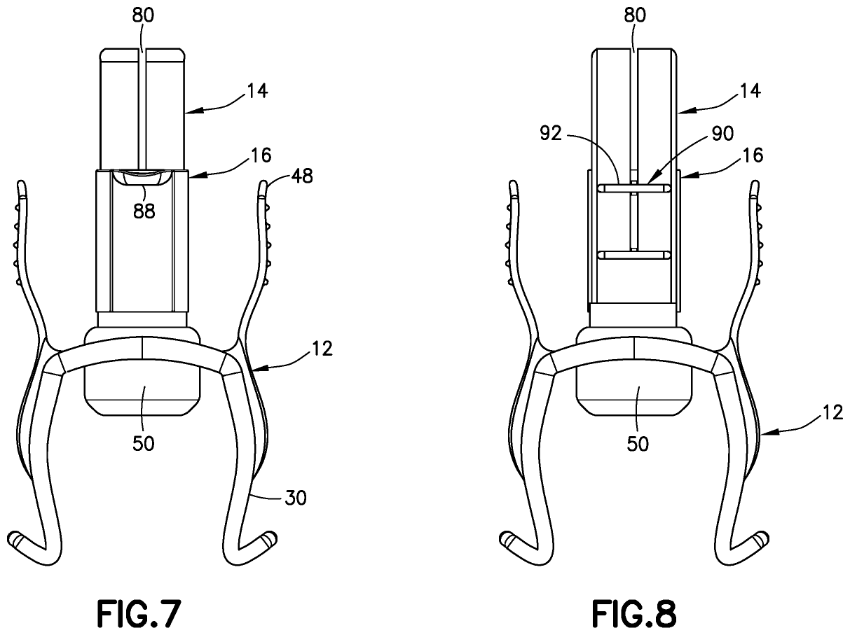

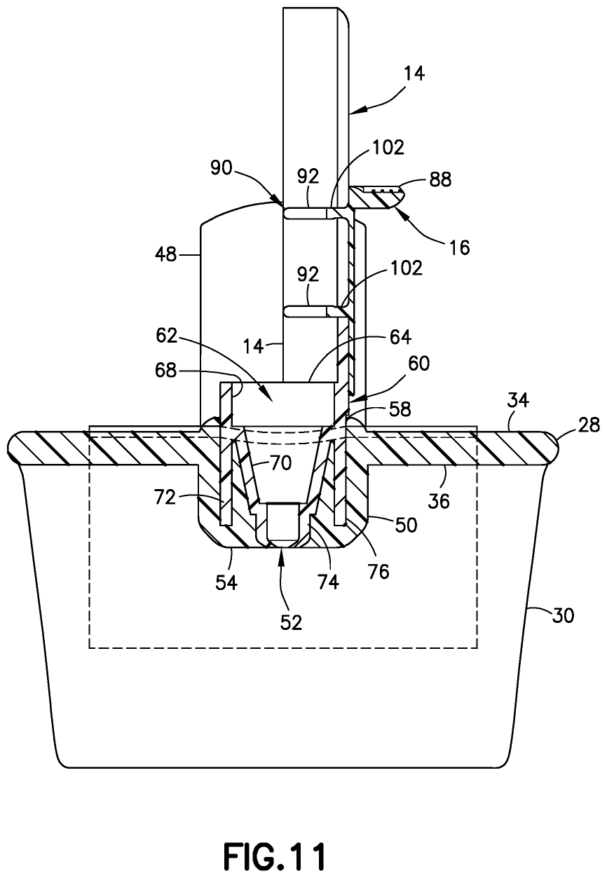

Referring to FIGS. 7 and 8, the body 28 has an axially collar 50 extending axially from the bottom side of the body toward the distal ends of the arms 30. The collar 50 defines an axial passage 52 and includes an inwardly extending portion 54 forming a distal face 56 as shown in FIG. 11. The distal face 56 has a dimension and orientation to form a contact surface for the surface of the skin and tissue during the pinching of the tissue and the injection. As shown in FIG. 11, the axial passage 52 extending through the body of the adapter 12 to an open end 58 at the top face of the adapter 12. The open end 58 has a dimension and configuration for receiving the guide 14. In the embodiment shown, the inner surface of the axial passage 52 has a shape corresponding to the guide 12 to allow the guide to fit within the open end.

The guide 14 as shown in the drawings can be formed as a separate member that can be coupled to the adapter 12. The guide 14 can be coupled to the adapter 12 by a friction fit or interference fit and can be removable from the adapter 12. In an alternative embodiment, the guide 14 can be fixed to the adapter 12, such as by an adhesive.

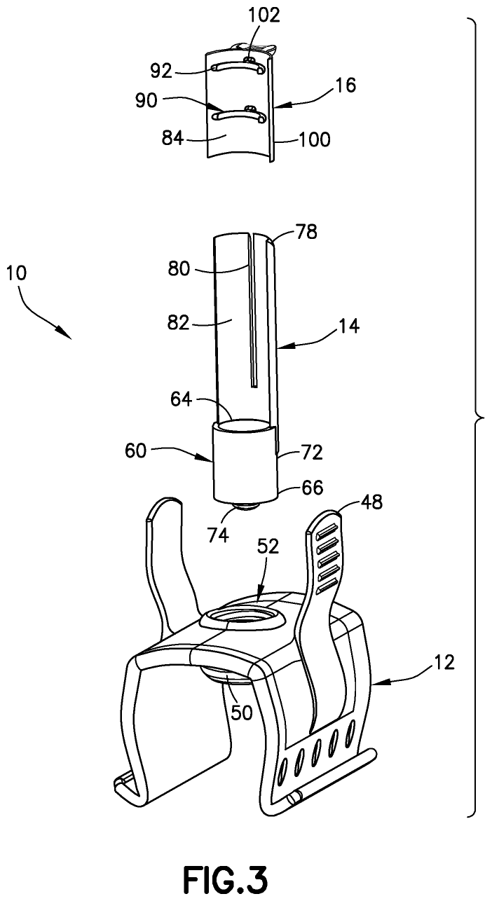

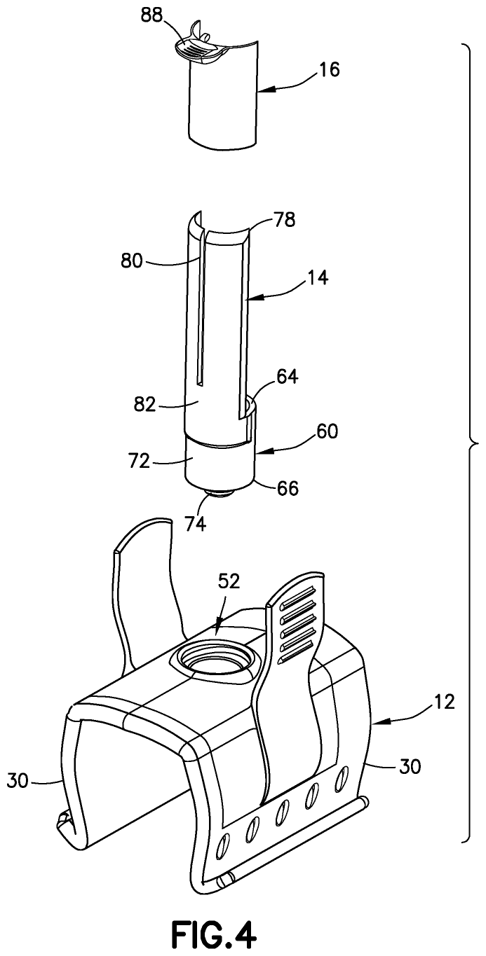

The guide 14 as shown in FIGS. 3, 4 and 11 has a distal end forming a base 60 having a cylindrical shape with an axial passage 62, an open top end 64, and an open bottom end 66. The inner surface 68 of base 60 has shape complementing the distal end of the syringe or other delivery device and is configured for receiving the distal end of the syringe. In the embodiment shown, the inner surface 68 at the bottom end of the base 60 has conical shaped surface 70 and a distally extending cylindrical sleeve 72 corresponding to the shape of the syringe. A cylindrical collar 74 extends distally from the base 60 that can be received in an annular recess 76 in the collar 50 of the adapter.

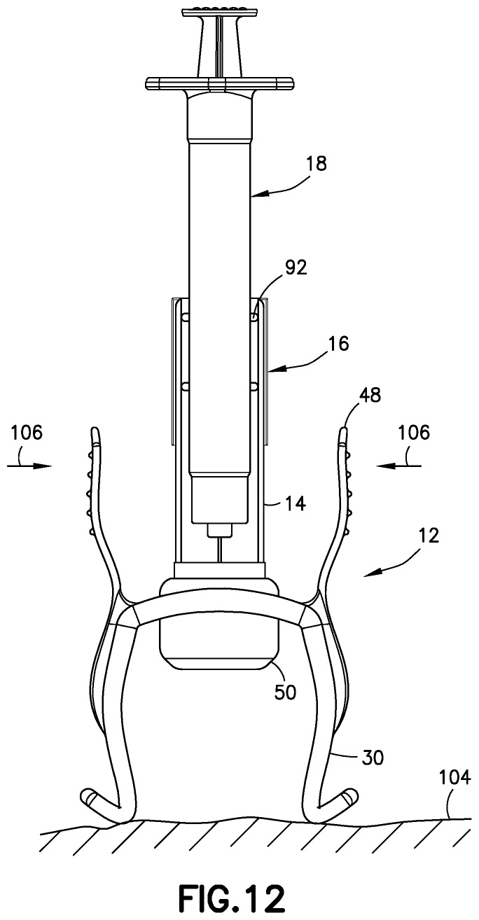

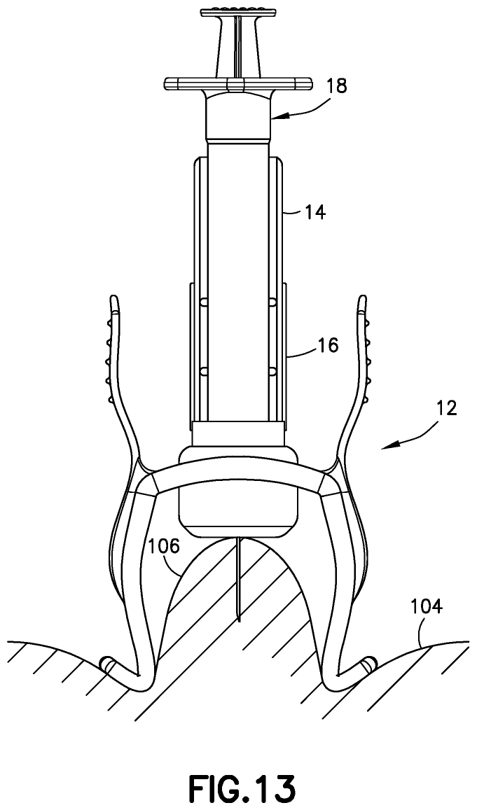

As shown in FIG. 3, the guide 14 has a proximally extending wall 78 having an opening. In the embodiment shown, the opening in the wall 78 is a longitudinally extending guide slot 80 for receiving the holder 16. The wall 78 has an inner surface 82 with a shape corresponding to the outer surface of the holder 16 where the holder and the delivery device, such as the syringe, can slide axially relative to the guide 14 and the adapter 12. The wall 78 has an axial length to enable the delivery device to move axially a distance where the needle of the delivery device is in a retracted position relative to the adapter shown in FIG. 12 and an extended position where the needle pierces the tissue of the patient as shown in FIG. 13.

The holder 16 as shown in FIG. 3 has body with shape complementing the shape of the wall 78 to slide axially on the guide 14. As shown, the holder has a curve shaped wall 100 with an inner surface 84 and an outer surface 86. An outwardly extending tab 88 projects from the outer surface 84 for manually moving the holder with respect to the guide 14 and the adapter 12.

The holder 16 includes a coupling for attaching the syringe or other delivery device to the holder for moving axially relative to the guide and adapter. The coupling in the embodiment shown includes a coupling member 90 configured to grip the syringe barrel or delivery device. The coupling member 90 as shown has curved generally open U-shape to grip the outer surface of the syringe barrel. The coupling member 90 includes two curved, flexible fingers 92 forming a semi-circular opening for gripping the syringe or pen needle delivery device. The fingers 92 are spaced inwardly from the inner surface 84 by a tab 102 a distance corresponding to the thickness of the wall 78 of the guide 14. As shown in FIGS. 2 and 3, the fingers 92 of the coupling are oriented inwardly with respect to the inner surface of the wall 78 of the guide 14 and the semi-circular wall of the holder is positioned on the outer side surface of the wall 78 in the assembled and ready to use position. As shown, the fingers 92 are positioned on an opposite side of the wall 78 from the wall 100 of the holder 16 with the tab 102 extending through the slot 80. The fingers 92 are able to grip the syringe 18 for moving the syringe in an axial direction to inject the medication, such as the insulin, to the patient.

During use, the syringe 18 or other delivery device is coupled to the holder 16 as shown in FIGS. 12 and 13 and the holder 16 is positioned in guide 14 in the adapter 12. The syringe is positioned with the needle 22 distal of the collar 50 so that needle is positioned in the collar 50 as shown in FIG. 12 in the ready position. The arms 30 are placed against the surface of the tissue or skin 104 as shown in FIG. 12. The tabs 48 are pressed inwardly in the direction of arrows 106 to spread the arms 30 outward to grip the surface of the tissue 104. The force applied to the tabs 48 is released so that the arms 30 spring back to the original position and pinch the tissue to form a raised area 108 as shown in FIG. 13. The holder 16 and the syringe 18 are manually pressed toward the raised area 108 of the tissue 104 where the needle 22 pierces the tissue to a depth determined by the travel of the holder relative to the adapter and the length of the needle. In one embodiment as shown, the arms of the adapter pinch the tissue so that the raised area contacts the distal end of the collar on the adapter to provide a controlled depth of penetration of the needle into the tissue.

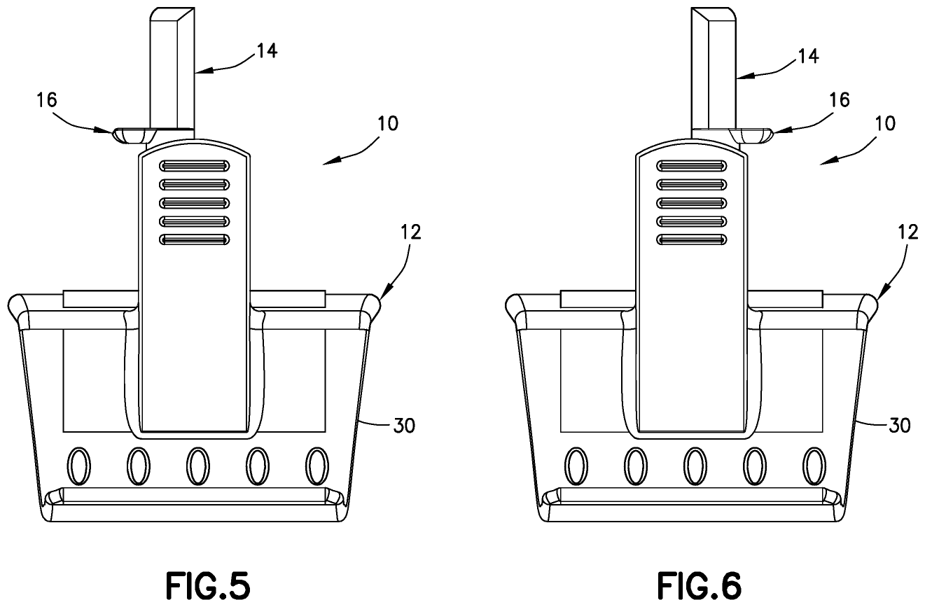

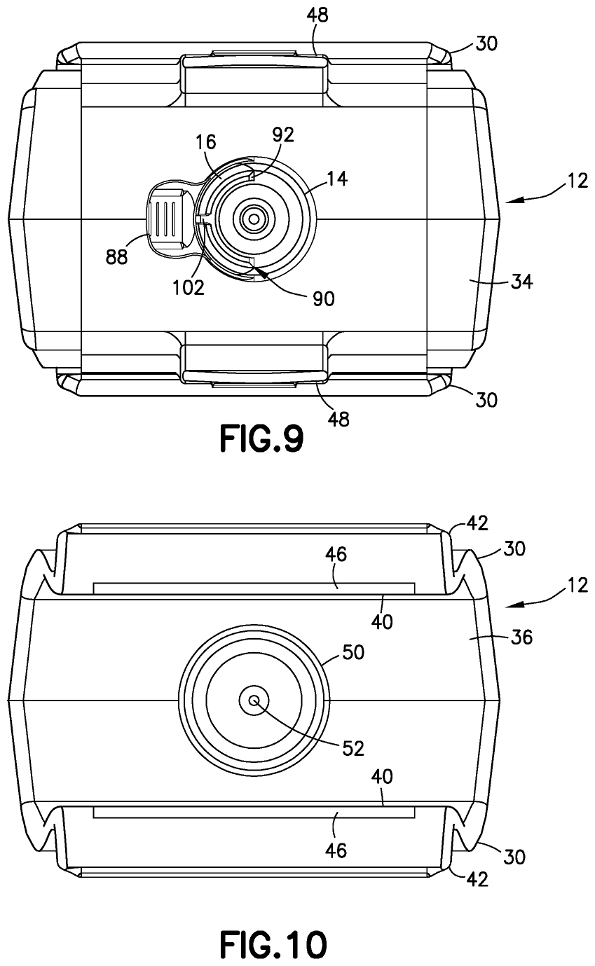

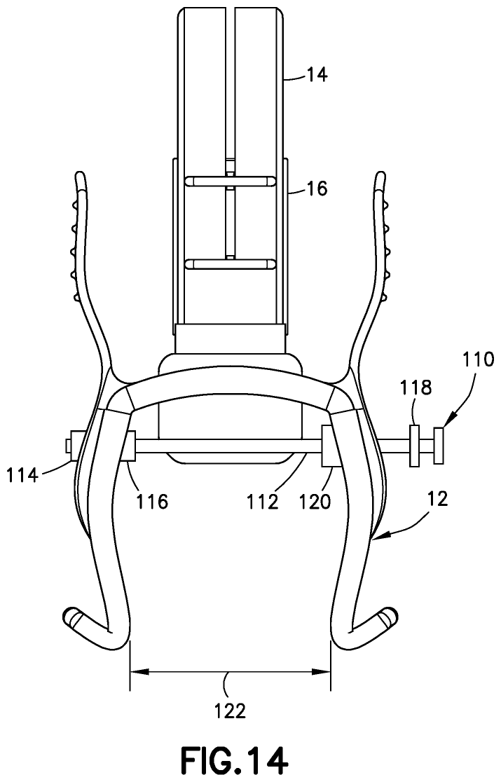

In the embodiment of FIGS. 1-13, the arms 30 are biased by the spring tension on the arms. The spacing of the distal end of the arms is determined by the shape of the adapter in the relaxed position. The spacing between the arms in the relaxed position determines the gathering or pinching of the tissue and height and dimension of the tissue for the injection. In another embodiment shown in FIG. 14, an adjustment mechanism 110 is provided to limit the opening and/or closing of the arms 30 to control the size of the gap between the distal end of the arms 30 for pinching of the tissue and the shape and dimension of the raised area of the tissue for the injection. In the embodiment shown in FIG. 14, the adjustment mechanism 110 is a threaded member 112, such as a threaded screw, that extends through the arms. A threaded stop member can be included on the threaded member to limit the distance that the arms can open and/or limit the distance between the arms when the tension is release to control the pinching and deformation of the tissue.

As shown in FIG. 14, a stop member 114 can be fixed to the distal end of the threaded member on an outer side of a first arm and a stop member 116 on an inner side to the arm. In one embodiment, the stop members can be fixed on the threaded member or can be adjustable. A stop member 118 on a proximal end of the threaded member can be provided on an outer side of the second arm and a stop member 120 can be provided on the inner side of the second arm. The stop members on the proximal end of the threaded member can be include internal threads to be adjustable on the threaded member 112 to limit the spacing between the arms in the rest position indicated by the arrow 122 shown in FIG. 14 and to limit the opening of the arms during use to control the amount of the surface of the tissue being gathered and pinched to control the shape and dimension of the raised portion of the tissue during the injection.

Although only a few embodiments of the present device are shown and described, the present device is not limited to the described embodiments. Instead, it will be appreciated by those skilled in the art that changes may be made to these embodiments without departing from the principles and spirit of the device. Different embodiments can be combined with other embodiments as long as they are not inconsistent with each other. It is particularly noted that those skilled in the art can readily combine the various technical aspects of the various elements of the various exemplary embodiments that have been described above in numerous other ways, all of which are considered to be within the scope of the disclosure and equivalents thereof.

1. A support for an injection delivery device comprising:
an adapter having at least two movable arms, each arm having a proximal end movable relative to each other and a distal end configured for engaging and pinching tissue of a patient to form a raised area of the tissue;
a guide coupled to said adapter and oriented between said at least two arms; and
a holder coupled to said guide, said holder configured for supporting the injection delivery device and for moving the injection delivery device in a distal direction relative to the holder and the adapter to deliver a medication to the tissue pinched between said arms.

2. The support of claim 1, wherein said injection delivery device has a needle extending in a distal direction for delivering the medication to the tissue pinched between said arms.

3. The support of claim 2, wherein said injection delivery device is a syringe.

4. The support of claim 1, wherein said adapter is a one-piece, unitary member having a body with said arms extending distally from said body, and where said arms are biased toward each other to pinch the tissue of the patient.

5. The support of claim 4, wherein said one-piece unitary member is a spring member.

6. The support of claim 4, wherein said body has an axial opening configured for receiving said guide, and where said holder is coupled to said guide for sliding axially and sliding said delivery device through said axial opening into contact with the tissue of the patient.

7. The support of claim 6, wherein said holder includes a coupling member for coupling to said delivery device for sliding said delivery device relative to said guide and adapter.

8. The support of claim 7, wherein said holder is configured to slide on an outer surface of said guide and said coupling member is configured to slide on an inner surface of said guide.

9. A support for an injection delivery device for delivering a medication to a patient, said support comprising:
an adapter having a body with an axial passage and two arms extending in a distal direction from said body, said adapter being a one-piece, unitary member, and where said arms are biased inwardly toward each other and said axial passage, said arms configured for pinching tissue of a patient between said arms;
a guide removably coupled to said adapter, said guide having an axial passage aligned with said axial passage of said adapter; and
a holder coupled to said guide, said holder having a coupling member configured for coupling to the injection delivery device, said holder configured for sliding said injection delivery device in an axial direction relative to said guide and adapter for introducing a needle of said injection delivery device into the tissue captured between said arms.

10. The support of claim 9, wherein said body of said adapter is made of a spring metal.

11. The support of claim 9, wherein each of said arms of said body include a distal end with a tab extending outwardly and proximally relative to said adapter.

12. The support of claim 11, wherein said body of said adapter has a distal face with a distally extending collar surrounding said axial passage and extending between said arms, and where said holder is received in said collar.

13. The support of claim 12, wherein said distally extending collar has a length to contact the tissue pinched between said arms.

14. The support of claim 13, wherein said guide has a distal end for engaging an inwardly extending flange at a distal end of said collar, and an inner sleeve extending at the distal end of the guide with an internal space for receiving a distal end of the delivery device where the needle of the delivery device can extend distally from said inner sleeve.

15. The support of claim 9, wherein said coupling member of said holder includes a flexible coupling member for coupling to the injection delivery device.

16. The support of claim 9, wherein said holder includes a body with an inner surface for sliding on an outer surface of said guide, and where said coupling member of said holder slides on an inner surface of said guide.

17. The support of claim 16, wherein said guide includes a longitudinally extending opening, and where said coupling member of said holder extends through said longitudinally extending opening.

18. A method of injecting a medication into subcutaneous tissue of a patient, said method comprising;
positioning a support against a patient, said support including an adapter having a body with an axial passage and two arms extending in a distal direction from said body, said adapter being a one-piece, unitary member, and where said arms are biased inwardly toward each other and configured for pinching tissue of a patient between said arms; a guide removably coupled to said adapter, said guide having an axial passage aligned with said axial passage of said adapter; and a holder coupled to said guide, said holder having a coupling member configured for coupling to the injection delivery device, said holder configured for sliding said injection delivery device in an axial direction relative to said guide and said adapter for introducing a needle of said injection delivery device into the tissue between said arms,
expanding the arms of the support outwardly and releasing the arms to pinch the subcutaneous tissue of the patient to form a pinched surface,
coupling the injection delivery device into the holder and guide, and coupling the holder and guide to the adapter,
sliding said holder and injection device relative to said guide and adapter to insert the needle into the subcutaneous tissue, and delivering the medication to the patient.

19. The method of claim 18, wherein said holder slides on an outer surface of the guide, and the coupling member of the holder slides on an inner surface of the guide.

20. The method of claim 19, wherein said guide includes a tab extending between said holder and said coupling member, and where said tab slides in a slot in the guide.
